(19) United States
(12) Patent Application Publication
(10) Pub. No.: US 2007/0100234 A1
(43) Pub. Date: May 3, 2007

(54) Methods and systems for tracking instruments in fluoroscopy

(76) Inventors: Jerome Stephen Arenson, Haifa (IL); David Ruimi, Netanya (IL); Oded Meirav, Haifa (IL); Haim E. Gelman, Migdal Haemek (IL)

Correspondence Address: PATRICK W. RASCHE (12553 - 1000), ARMSTRONG TEASDALE LLP, ONE METROPOLITAN SQUARE, SUITE 2600, ST. LOUIS, MO, 63102-2740, US

(21) Appl. No.: 11/260,056

(22) Filed: Oct. 27, 2005

(51) Int. Cl. A61B 5/05

(52) U.S. Cl. 600/429

(57) ABSTRACT

Methods and systems for displaying an instrument in a region of interest are provided. The imaging system includes a multi-slice detector, a processor coupled to the multi-slice detector, and a display configured to display reconstructed images. The processor is configured to receive a plurality of multi-slice scan data, identify at least a portion of an instrument in at least one slice of the plurality of multi-slice scan data, and display the identified instrument portion with an indicator associated with the at least one slice.

### BACKGROUND OF THE INVENTION

This invention relates generally to computed tomography (CT) imaging and more particularly, to tracking instruments during interventional CT Fluoroscopy.

In at least one known CT system configuration, an x-ray source projects a fan-shaped beam which is collimated to lie within an X-Y plane of a Cartesian coordinate system and generally referred to as the “imaging plane” The x-ray beam passes through the object being imaged, such as a patient. The beam, after being attenuated by the object, impinges upon an array of radiation detectors. The intensity of the attenuated beam radiation received at the detector array is dependent upon the attenuation of the x-ray beam by the object. Each detector element of the array produces a separate electrical signal that is a measurement of the beam attenuation at the detector location. The attenuation measurements from all the detectors are acquired separately to produce a transmission profile.

In known third generation CT systems, the x-ray source and the detector array are rotated with a gantry within the imaging plane and around the object to be imaged so that the angle at which the x-ray beam intersects the object constantly changes. A group of x-ray attenuation measurements, i.e., projection data, from the detector array at one gantry angle is referred to as a “view” A “scan” of the object comprises a set of views made at different gantry angles, or view angles, during one revolution of the x-ray source and detector. In an axial scan, the projection data is processed to construct an image that corresponds to a two dimensional slice taken through the object. One method for reconstructing an image from a set of projection data is referred to in the art as the filtered back projection technique. This process converts the attenuation measurements from a scan into integers called “CT numbers” or “Hounsfield units,” which are used to control the brightness of a corresponding pixel on a display device.

To reduce the total scan time, a “helical” scan may be performed. To perform a “helical” scan, the patient is moved while the data for the prescribed number of slices is acquired. Such a system generates a single helix from a one fan beam helical scan. The helix mapped out by the fan beam yields projection data from which images in each prescribed slice may be reconstructed.

Reconstruction algorithms for helical scanning typically use helical weighting (“HW”) algorithms which weight the collected data as a function of view angle and detector channel index. Specifically, prior to filtered back projection, the data is weighted according to a helical weighting factor, which is a function of both the view angle and detector angle. As with underscan weighting, in a HW algorithm, projection data is filtered, weighted, and backprojected to generate each image.

In multi-slice CT fluoroscopy, a fan beam of x-rays is projected toward a detector that includes a plurality of rows of detector elements in the z-axis direction. Each row of detector elements is used to reconstruct an image of a target lying between the source of the x-ray beam and the detector. Any number of images may be combined to generate a volumetric image of the target and/or sequential frames of images to help, for example, in guiding a needle to a desired location within a patient. A frame, like a view, corresponds to a two dimensional slice taken through the imaged object. Particularly, projection data is processed at a frame rate to construct an image frame of the object.

In CT Fluoro systems, it is generally advantageous to increase the frame rate while minimizing image degradation. Increasing the frame rate provides advantages including, for example, that an operator physician is provided with more timely (or more up to date) information regarding the location of, for example, a biopsy needle. However, increasing the frame rate generally adversely affects minimizing image degradation. For example, an increase in the frequency that projection data is filtered, weighted and backprojected, tends to slow the frame rate. The frame rate is thus limited to the computational capabilities of the CT Fluoro system. As the number of acquired slices per gantry rotation offered in multi-slice CT systems increases, the user is unable to use all the information available. More specifically, in interventional CT procedures the user is challenged when attempting to monitor multi-slice scanners which are capable of presenting multiple images at frame rates often exceeding approximately 10 frames per second. With multi-slice CT Fluoro systems, one to three thick-slice summations of the available thinner axial slices can be presented as summed images, however, such a summation foregoes the potential resolution enhancement afforded by thin slice imaging. As a result, such summation may not provide the possible improved needle placement accuracy afforded by multi-slice scanners.

Additionally, the trajectory of the needle insertion during the interventional procedure (biopsy, drainage etc.) may be different from the axial plane such that in conventional CT single-slice interventional procedures, the needle insertion is generally limited to the image plane only and any Z direction needle position change requires patient table movement in the appropriate direction. The decision regarding the correct magnitude and direction of this movement requires experience and frequently involves a “trial and error” approach. Moreover, there is an added risk of moving the patient table and patient In and Out of the gantry aperture during the procedure while the needle remains inserted in the patient's body.

### BRIEF DESCRIPTION OF THE INVENTION

In one embodiment, an imaging system for displaying an instrument in a region of interest is provided. The imaging system includes a multi-slice detector, a processor coupled to the multi-slice detector, and a display configured to display reconstructed images. The processor is configured to receive a plurality of multi-slice scan data, identify at least a portion of an instrument in at least one slice of the plurality of multi-slice scan data, and display the identified instrument portion with an indicator associated with the at least one slice.

In another embodiment, a computer system is provided. The computer system is configured to receive a plurality of multi-slice scan data, and identify at least a portion of a needle-like instrument positioned in at least one slice of the multi-slice scan data with an indicator associated with the slice.

In yet another embodiment, a method of displaying an instrument in a region of interest is provided. The method includes associating an indicator including at least one of a color, a shading, and a pattern with each slice of a multi-slice image of a region of interest, identifying at least a portion of an instrument in at least one slice, and applying the indicator associated with the slice, to the identified instrument portion in that slice.

In still another embodiment, an imaging scanner is provided. The imaging scanner includes a data acquisition apparatus configured to acquire imaging data from a subject, a monitor configured to display images reconstructed from the acquired imaging data and a computer programmed to acquire multiple slices of imaging data from the subject having an intracorporeal device positioned therein, reconstruct a multi-slice image from the multiple slices of imaging data, and cause the monitor to display the multi-slice image at a real-time frame rate while preserving information of a position of the intracorporeal device contained in the multiple slices of imaging data for observation by a human observer.

In another embodiment, a method of tracking an invasive instrument relative to a target using an imaging system that includes a movable patient table and a multi-slice detector array to automatically move the scan plane of the imaging system within the Z coverage area of the multi-slice detector array is provided. The method includes determining an intracorporeal trajectory of the instrument, displaying a tip of the instrument in at least one of a plurality of adjacent slices, and translating a patient table when the tip reaches a substantial extent of the Z coverage area.

### BRIEF DESCRIPTION OF THE DRAWINGS

FIG. 1 is a pictorial view of a multi-slice volumetric CT imaging system;

FIG. 2 is a block schematic diagram of the multi-slice volumetric CT imaging system illustrated in FIG. 1;

FIG. 3 is a flow chart of an exemplary method of displaying an instrument in a region of interest;

FIG. 4 is an exemplary CT fluoroscopy scan image that includes a region of interest;

FIG. 5 is another exemplary CT fluoroscopy scan image that includes the region of interest shown in FIG. 4;

FIG. 6 is an exemplary display that may be output through the display shown in FIG. 2;

FIG. 7 is a side schematic view of an embodiment of the patient table that may be used with the imaging system shown in FIG. 1;

FIG. 8 is a flow diagram of an exemplary method of a tracking algorithm to automatically move the scan plane within the Z coverage of the multi-slice detector array; and

FIG. 9 is a exemplary CT fluoroscopy scan image area that includes a region of interest described in method in FIG. 8.

### DETAILED DESCRIPTION OF THE INVENTION

As used herein, an element or step recited in the singular and preceded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Also as used herein, the phrase, “reconstructing an image” is not intended to exclude embodiments of the present invention in which data representing an image is generated but a viewable image is not. Therefore, as used herein the term, “Image,” broadly refers to both viewable images and data representing a viewable image. However, many embodiments generate (or are configured to generate) at least one viewable image. Additionally, although described in detail in a CT medical setting, it is contemplated that the benefits accrue to all imaging modalities including, for example, ultrasound, Magnetic Resonance Imaging, (MRI), Electron Beam CT (EBCT), Positron Emission Tomography (PET), Single Photon Emission Computed Tomography (SPECT), and in both medical settings and non-medical settings such as an industrial setting or a transportation setting, such as, for example, but not limited to, a baggage scanning CT system for an airport or other transportation center.

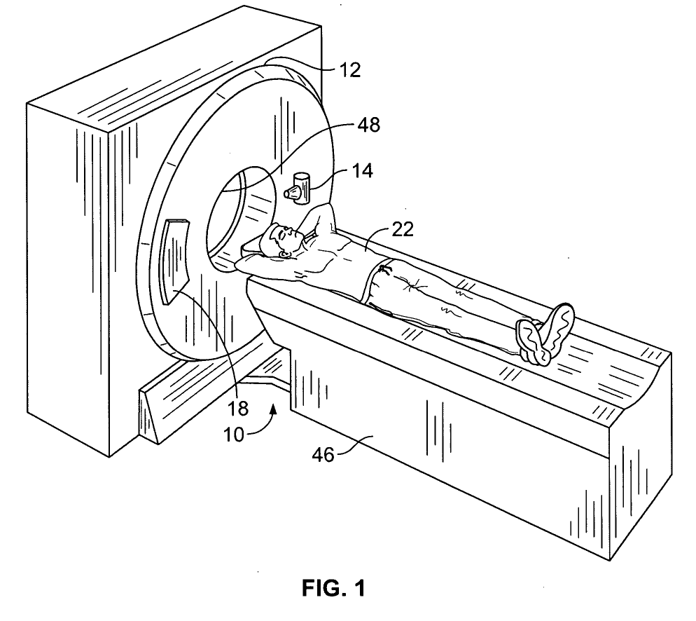

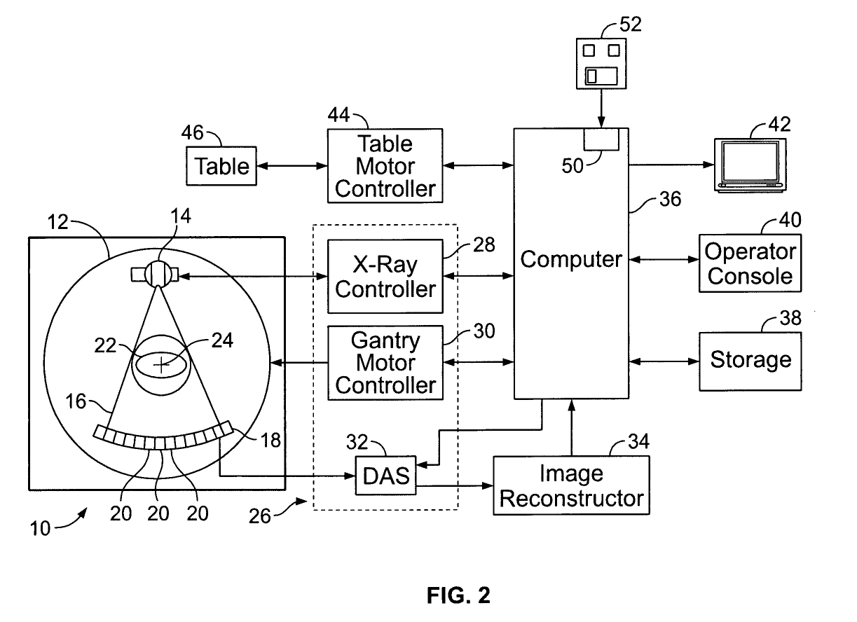

FIG. 1 is a pictorial view of a CT imaging system 10. FIG. 2 is a block schematic diagram of system 10 illustrated in FIG. 1. In the exemplary embodiment, a computed tomography (CT) imaging system 10 is shown as including a gantry 12 representative of a “third generation” CT imaging system. Gantry 12 has a radiation source 14 that projects a cone beam 16 of X-rays toward a detector array 18 on the opposite side of gantry 12.

Detector array 18 is formed by a plurality of detector rows (not shown) including a plurality of detector elements 20 which together sense the projected X-ray beams that pass through an object, such as a medical patient 22. Each detector element 20 produces an electrical signal that represents the intensity of an impinging radiation beam and hence the attenuation of the beam as it passes through object or patient 22. An imaging system 10 having a multi-slice detector 18 is capable of providing a plurality of images representative of a volume of object 22. Each image of the plurality of images corresponds to a separate “slice” of the volume. The “thickness” or aperture of the slice is dependent upon the thickness of the detector rows.

During a scan to acquire radiation projection data, gantry 12, and the components mounted thereon rotate about a center of rotation 24. FIG. 2 shows only a single row of detector elements 20 (i.e., a detector row). However, multi-slice detector array 18 includes a plurality of parallel detector rows of detector elements 20 such that projection data corresponding to a plurality of quasi-parallel or parallel slices can be acquired simultaneously during a scan.

Rotation of gantry 12 and the operation of radiation source 14 are governed by a control mechanism 26 of CT system 10. Control mechanism 26 includes a radiation controller 28 that provides power and timing signals to radiation source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized radiation data from DAS 32 and performs high-speed image reconstruction. The reconstructed image is applied as an input to a computer 36 which stores the image in a mass storage device 38.

Computer 36 also receives commands and scanning parameters from an operator via console 40 that has a keyboard. An associated display 42, for example, a monitor, allows the operator to observe the reconstructed image and other data from computer 36. The operator supplied commands and parameters are used by computer 36 to provide control signals and information to DAS 32, radiation controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

In one embodiment, computer 36 includes a device 50, for example, a floppy disk drive or CD-ROM drive, for reading instructions and/or data from a computer-readable medium 52, such as a floppy disk or CD-ROM. In another embodiment, computer 36 executes instructions stored in firmware (not shown). Generally, a processor in at least one of DAS 32, reconstructor 34, and computer 36 shown in FIG. 2 is programmed to execute the processes described below. Of course, the method is not limited to practice in CT system 10 and can be utilized in connection with many other types and variations of imaging systems. In one embodiment, computer 36 is programmed to perform functions described herein, accordingly, as used herein, the term computer is not limited to just those integrated circuits referred to in the art as computers, but broadly refers to computers, processors, microcontrollers, microcomputers, programmable logic controllers, application specific integrated circuits, and other programmable circuits.

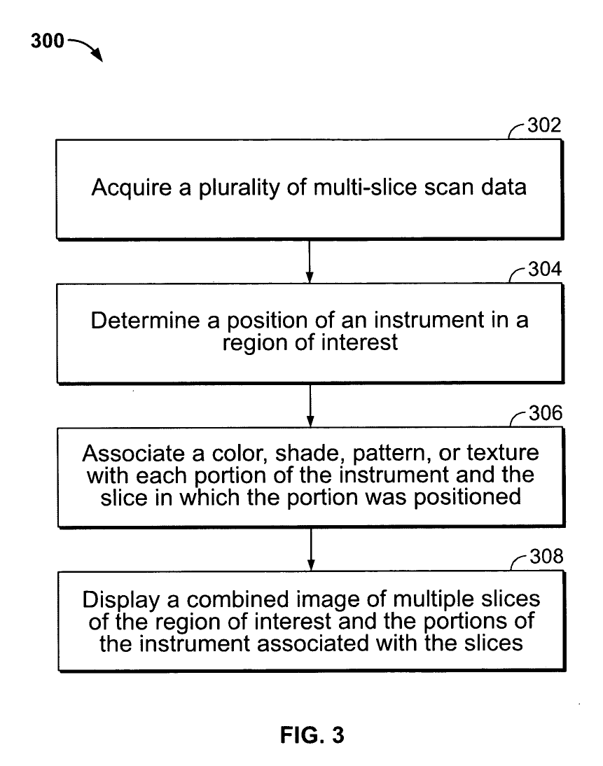

FIG. 3 is a flow chart of an exemplary method 300 of displaying an intracorporeal device, such as a medical instrument in a region of interest. The method includes acquiring 302 a plurality of multi-slice scan data. Each slice of the multi-slice scan is analyzed and the portion of the instrument included in each slice is identified. Identification is performed automatically by any of a number of techniques, for example, but not limited to, an image threshold detection based on the relatively high CT values of the instrument, for example, a metallic needle, and/or techniques such as image analysis or preprocessed sinogram data analysis based on pre-designated entrance and target locations. Using such analyses a position of the instrument is determined 304 within the region of interest with respect to each slice of the multi-slice scan data.

In the exemplary embodiment, each thin slice of an n-slice multi-slice scanner is designated a specific indicator, such as a color, a shade, a pattern, or a texture that is chosen such that a natural continuum of n colors corresponds to the n detector rows. The selected continuum could be, for example, a heat spectrum, a rainbow, or other progression of colors. Similarly, a continuum of shading, patterns or textures may be associated with each detector row. Associating elements of the continuum is performed on a slice by slice basis, where segments or portions of the instrument that appear in the slice are assigned the appropriate element for the selected continuum. In one embodiment, for example, a rainbow spectrum is selected as the continuum for a color indicator for a biopsy needle instrument. In a rainbow spectrum the colors transition from red, orange, yellow, green, blue, indigo and violet. The colors are not discrete bands of color, rather the colors transition continually from violet to red. In the case where six slices are used to reconstruct the image of the region of interest, each slice is assigned a color based on the selected continuum. In the example of the rainbow spectrum, a first slice at one end of the region of interest is assigned red, an adjacent slice is assigned the color orange, the next adjacent slice is assigned the color yellow, and so on to the other end of the region of interest. A portion of the biopsy needle that is located in each slice is colorized the same color as the color assigned to the slice. Accordingly, a color, shade, pattern, or texture is associated 306 with each portion of the instrument and the slice in which the portion was positioned.

In the exemplary embodiment, an image of the region of interest is reconstructed using a plurality of the image slices from the multi-slice scan data. An image of the instrument, colorized in colors associated with each slice where the portion of the instrument was located is reconstructed. A combined image of multiple slices of the region of interest and the portions of the instrument associated with the slices is then displayed 308.

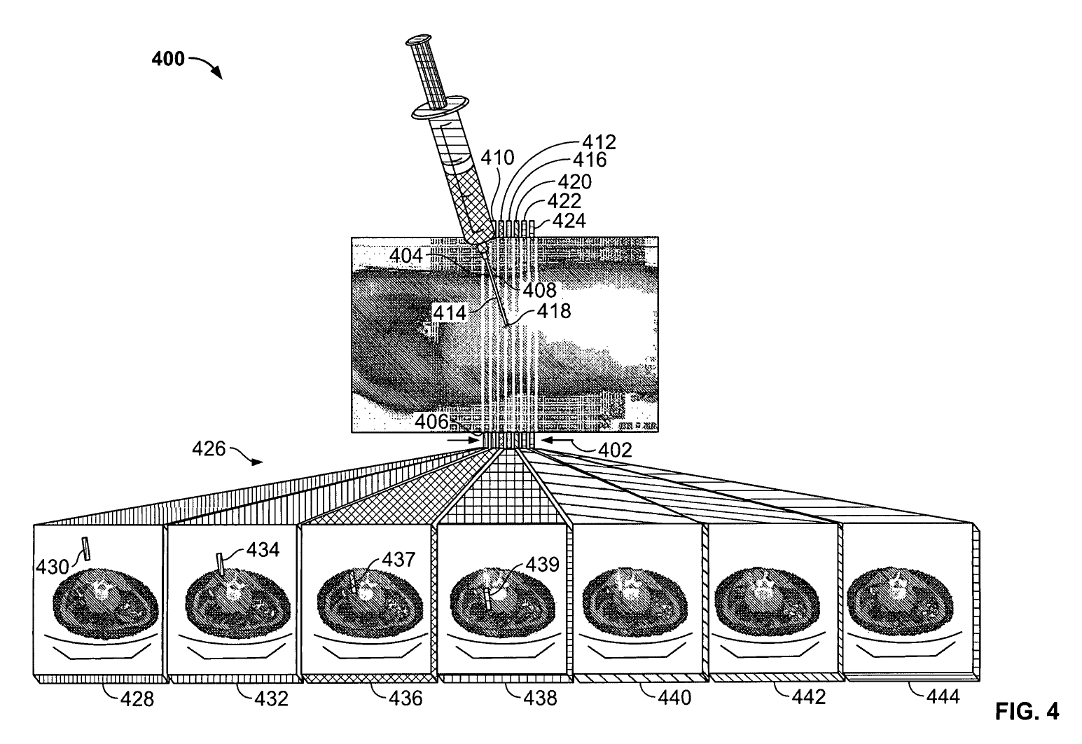

FIG. 4 is an exemplary CT fluoroscopy scan image area 400 that includes a region of interest 402. A medical instrument, such as a biopsy needle 404 is positioned within region of interest 402 during a procedure. A plurality of image slices of a cross section of region of interest 402 includes a portion of needle 404. In the exemplary embodiment, a slice 406 at a first end of region of interest 402 includes a base portion 408 of needle 404, a slice 410, and a slice 412 include portions of needle 404 that pass through each slice, and a slice 416 near the center of region of interest 402 includes a tip portion 418 of needle 404. Slices 420, 422, and 424 do not include any portion of needle 404. In the exemplary embodiment, each slice is associated with a different color of a selectable color spectrum continuum 426. For example, slice 406 is associated with red, slice 410 with red-orange, slice 412 with orange, slice 416 with yellow, slice 420 with light green, slice 422 with green, and slice 424 with blue. In various embodiments of the present invention, other selected spectrums and/or indicators will yield different colors, shading, pattern, or texture associated with each of slices 406, 410, 412, 416, 420, 422, and 424.

An image 428, reconstructed from the scan data associated with slice 406 includes an image portion 430 of needle 404. Portion 430 is colorized red, the color associated with the slice in which it is positioned. An image 432, reconstructed from the scan data associated with slice 410 includes an image portion 434 of needle 404. Portion 434 is colorized red-orange, the color associated with the slice in which it is positioned. Images 436 through 444 are likewise reconstructed from the scan data associated with scan data for slices of region of interest 402. Each of images 436 through 444 only includes a portion of needle 404 that is positioned within that slice. For example, image 436 includes an image portion 437 of needle 404 and image 438 includes an image portion 439 that illustrates tip 418 of needle 404. If needle 404 is not positioned such that any portion of needle 404 is located within a slice, the image corresponding to that slice will not include a portion of needle 404 in the image. For example, images 440, 442, 444 do not include a corresponding portion illustrating a position of needle 404 because needle 404 is not positioned such that a portion of needle 404 is located within the slice corresponding to images 440, 442, 444.

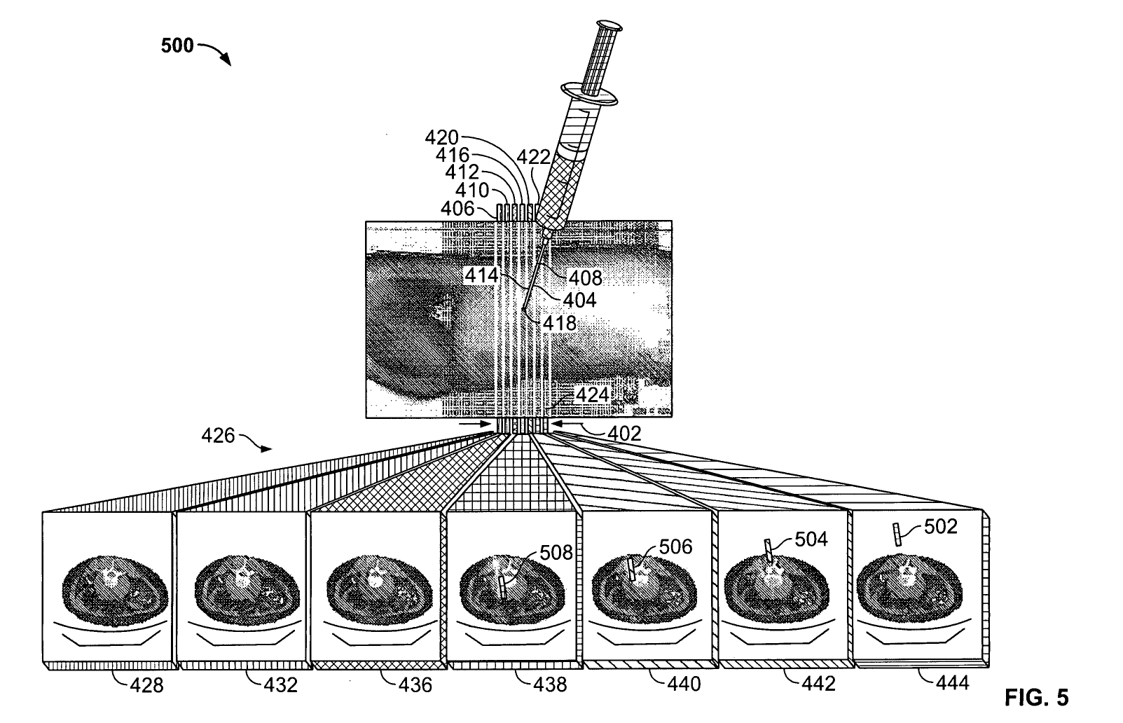

FIG. 5 is another exemplary CT fluoroscopy scan image area 500 that includes region of interest 402 shown in FIG. 4. Biopsy needle 404 is positioned within region of interest 402 during a procedure. In the exemplary embodiment, needle 404 is positioned such that tip 418 is located within slice 416 as shown in FIG. 4, except that needle 404 enters region of interest 402 from a different location than that shown in FIG. 4. A plurality of image slices of a cross section of region of interest 402 include a portion of needle 404. In the exemplary embodiment, slice 424, at a second end of region of interest 402, includes base portion 408 of needle 404, slice 422 and slice 420 include portions of needle 404 that pass through each slice, and slice 416, near the center of region of interest 402, includes tip portion 418 of needle 404. Slices 412, 410, and 406 do not include any portion of needle 404. In the exemplary embodiment, each slice is associated with a different color of a selectable color spectrum continuum 426 as illustrated above with regard to FIG. 4. Slice 406 is associated with red, slice 410 with red-orange, slice 412 with orange, slice 416 with yellow, slice 420 with light green, slice 422 with green, and slice 424 with blue.

Image 444, reconstructed from the scan data associated with slice 424 includes an image portion 502 of needle 404. Portion 502 is colorized blue, the color associated with the slice in which it is positioned. Image 442, reconstructed from the scan data associated with slice 422 includes an image portion 504 of needle 404. Portion 504 is colorized green, the color associated with the slice in which it is positioned. Images 428 through 440 are likewise reconstructed from the scan data associated with scan data for slices of region of interest 402. Each of images 428 through 440 only includes a portion of needle 404 that is positioned within that slice. For example, image 440 includes an image portion 506 of needle 404 and image 438 includes image portion 508 that illustrates tip 418 of needle 404. If needle 404 is not positioned such that any portion needle 404 is located within a slice, the image corresponding to that slice will not include a portion of needle 404 in the image. Accordingly, images 436, 432, and 428 do not include a corresponding portion illustrating a position of needle 404 because needle 404 is not positioned within the slice corresponding to images 436, 432, and 428.

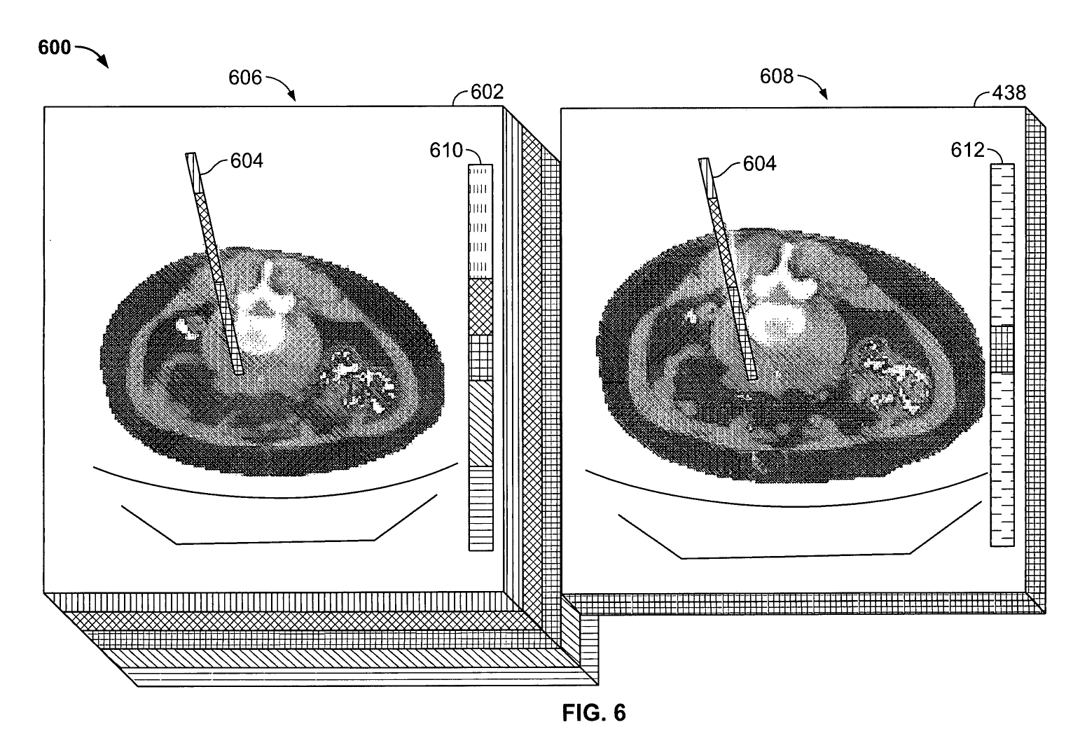

FIG. 6 is an exemplary display 600 that may be output through display 42 (shown in FIG. 2). A multi-slice relatively thicker image 602 includes an image comprising a plurality of slices. A composite view 604 of needle 404 is displayed as needle segments along with their proper color coding that are combined into a single multi-color needle shaft (if it passes through adjacent slices) whose orientation can be instantly understood. For example, as illustrated in FIG. 4, if red-orange-yellow-green-blue is assigned to the cranial-caudal slices, then a needle tip that is blue indicates a needle trajectory towards the feet, while a red tip indicates needle 404 is positioned towards the head. A yellow needle tip designates that it is positioned substantially in the middle of region of interest 402.

The viewer is presented a first viewing area 606 including single composite thick slice image 602 that is comprised of a combination, such as a summation, of the acquired n thin slices and overlayed with the multi-color composite needle segments. In the exemplary embodiment, this single composite slice is updated at high frame rates for observer viewing.

Improved placement information may be obtained by displaying a second viewing area 608 that includes a thin-slice image, for example, image 438 showing the needle tip, alongside combined thick slice image 602. Second viewing area 608 provides the viewer with a detailed, thin-slice, high-resolution image for confirmation of needle tip positioning. Automatic needle-tip identification and tracking may be accomplished in a similar fashion to the techniques described above.

In another embodiment, a third viewing area (not shown) displays a second thin-slice image, selected to lie in the plane of the target anatomy. This allows the observer to further confirm that needle 404 has reached the target.

A legend 610 indicates relative positions of the slices associated with each color, texture, or pattern used in composite thick slice image 602. Another legend 612, displayed with the thin slice image selected in second viewing area 608 illustrates the relative position of the portion of needle 404 associated with the slice selected and displays the needle portion in the color, texture, or pattern associated with that slice to facilitate confirmation of the position of needle 404 in any portion of region of interest 402.

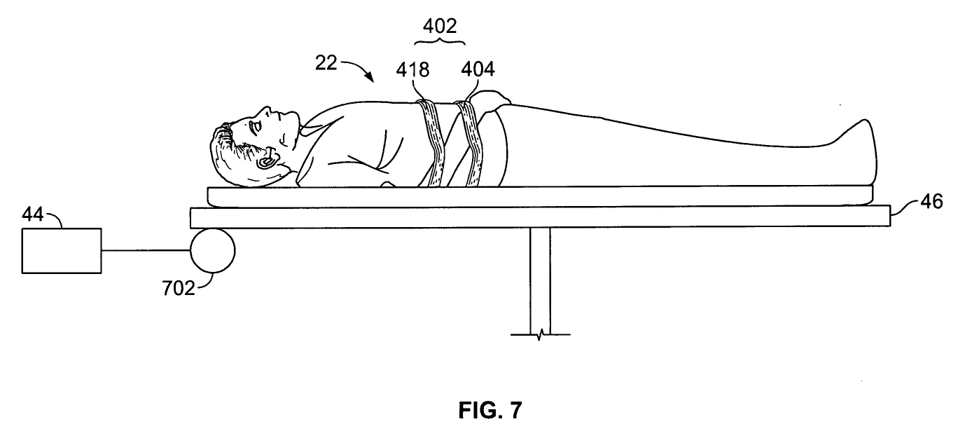

FIG. 7 is a side schematic view of an embodiment of patient table 46 that may be used with imaging system 10 (shown in FIG. 1). In the exemplary embodiment, patient 22 is lying on patient table 46 that includes a positioning motor 702 communicatively coupled to table motor controller 44 that automatically positions table 46 such that needle-tip 418 and region of interest 402 always lie in or near the central slice of system 10. Identification of needle tip 418 is performed automatically by any of a number of techniques, for example, but not limited to, an image threshold detection based on the relatively high CT values of the needle, and/or techniques such as image analysis or preprocessed sinogram data analysis based on pre-designated entrance and target locations. When needle tip 418 is identified, a command is sent to table motion controller 44 to reposition table 46 such that needle tip 418 is aligned with a central portion of display 42. Such needle-tracking is particularly appropriate where the needle insertion is significantly skewed to the axial plane, accordingly, such a method potentially permits needle insertion while maintaining the user's hands substantially outside of the x-ray beam.

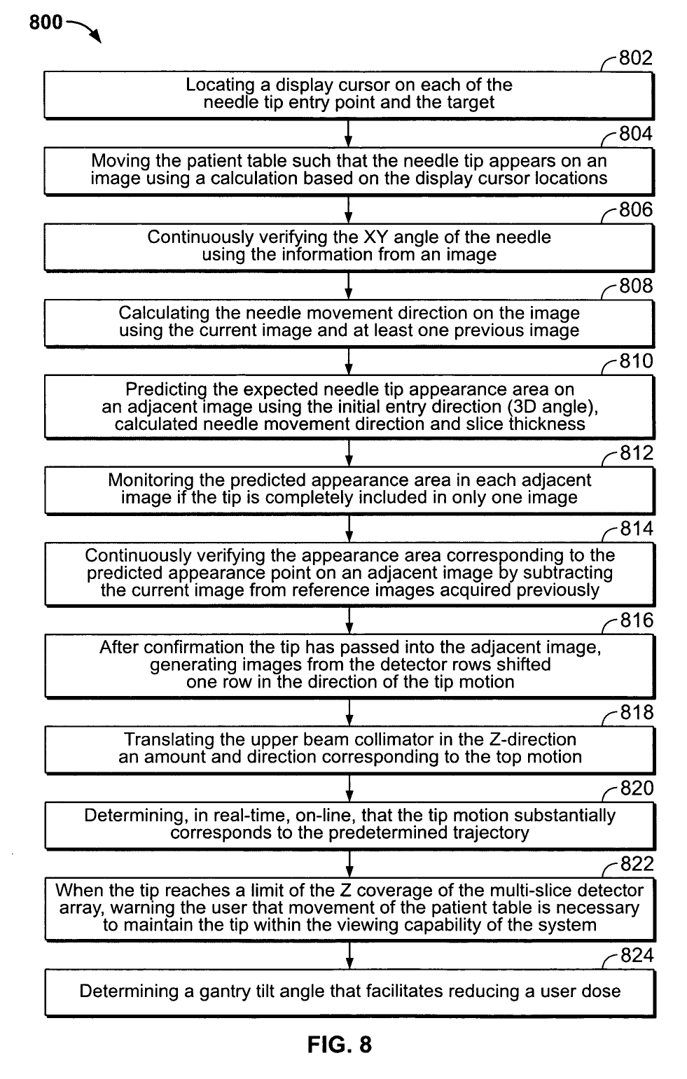

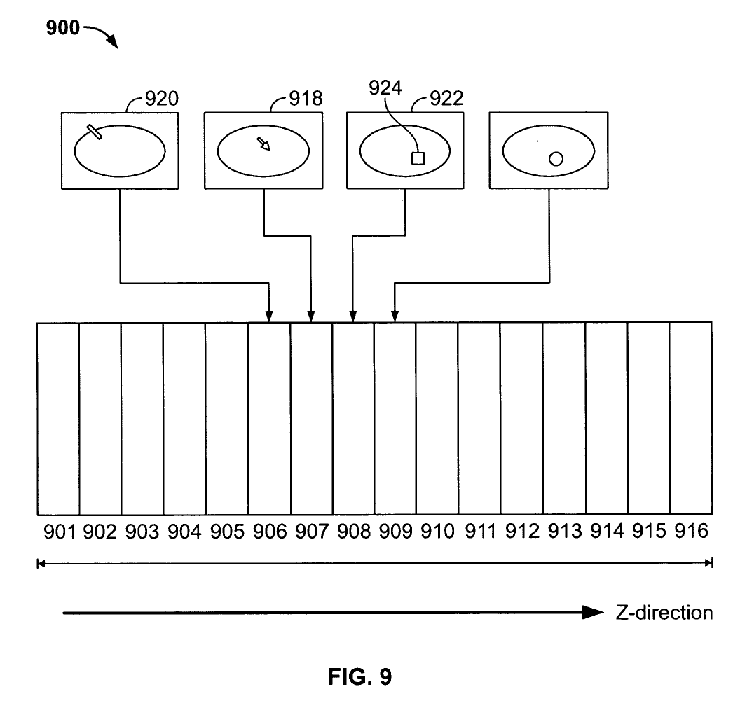

FIG. 8 is a flow diagram of an exemplary method 800 of a tracking algorithm to automatically move the scan plane within the Z coverage of the multi-slice detector array rather than moving the patient table to follow the needle tip. FIG. 9 is an exemplary CT fluoroscopy scan image area 900 that includes a region of interest described in method 800 in FIG. 8. The acquired data is analyzed using the attenuation information from one or more reconstructed images, raw data and/or preprocessed data to substantially determine the exact needle position. The reconstructed image displaying the needle tip will slide automatically according to the needle tip movement and the upper beam collimator will automatically track the needle tip movement in the Z direction, in order to reduce the patient and the operator dose. In the exemplary embodiment, the region of interest is represented by sixteen images, such as detector rows 901-916, each image corresponding to a slice of a sixteen slice detector.

Based on a previously performed volume scan, the user locates 802 a display cursor on each of a needle tip entry point and a target. These two points may be located at different table positions (images) to determine the planned needle trajectory.

The system moves 804 the patient table such that the needle tip appears on an image, for example, an image 918 using a calculation based on the display cursor locations. In the exemplary embodiment, the initial entry direction (3D angle) of the needle is adjusted by the user using a guide (i.e. laser, calipers, lights, etc.). In an alternative embodiment, tuning of the initial entry angle is based on acquiring continuous or “tap” scanning with very low dose of the needle out of the patient just prior to insertion into the patient. The calculation is based on at least two images wherein the images are based on data acquired by more than one detector row.

The XY angle of the needle is continuously verified 806 based on the information from image 920. The angle relative to the Z-axis is continuously verified based on the information from image 918 and image 920.

The needle movement direction is calculated 808 on image 918 by continuously subtracting the actual (current) and previous images 918. If the needle movement is slow, and the frame rate is fast, then the subtraction is performed on images 918 with longer time gaps.

Based on the initial entry direction (3D angle), calculated needle movement direction and slice thickness, the expected needle tip appearance area 924 on image 922 is predicted 810. If the needle is completely included in only one image, each adjacent image, for example, image 920 and image 922 are both monitored 812 in their predicted areas. These areas will be located adjacent to the needle tip position on image 918.

The area corresponding to the predicted appearance point on an image 922 is continuously verified 814 by subtracting the actual (current) image 922 from reference images 922 acquired previously. Verification that the needle tip has reached image 922 is confirmed by observing a dramatic density change within the predicted appearance area and/or confirmation of the density change for several consecutive reconstructed images. In the specific case where the needle is rigid, straight and has a relatively small angle (relative to z axis), the two adjacent images 920 and 922 may be sufficient for monitoring the needle positioning and predicted areas 918. For curved interventional tools the calculation can be done using thinner slice thicknesses and enlarging the predicted appearance areas 918.

After the confirmation, the system generates 816 images from rows 907, 908, 909, and 910 instead of rows 906, 907, 908, and 909 and the needle tip will remain in the displayed image 907 as before and the upper beam collimator translates 818 in the Z-direction a corresponding amount and direction.

The system verifies 820, in real-time, on-line, that the needle is traveling along the predetermined trajectory. If the needle deviates substantially from the predetermined trajectory by exceeding a selectable position threshold, a warning is indicated to the user. Such a warning is advantageous for procedures where the needle trajectory and the target area are not in the same imaged plane.

When the needle tip reaches 822 a limit of the Z coverage of the multi-slice detector array, for example, by exiting the last slice of the array, the user is warned that movement of the patient table, either manually or automatically, is necessary to maintain the needle tip within the viewing capability of the system.

Because the needle is able cross more than one slice plane (i.e. the needle is skewed to the scanner's axial plane), a significant dose saving to the user may be achieved by, for example, tilting the gantry. The system is programmed to determine 824 a recommended optimum gantry tilt angle for the specific interventional procedure used.

The above-described embodiments of an imaging system provide a cost-effective and reliable means for displaying wide scan coverage imaging while maintaining thin-slice detailed imaging for medical instrument insertion accuracy. More specifically, the needle color-coding provides a single thick-slice image while still showing thin-slice needle positioning to facilitate simultaneously benefiting from both aspects of multi-slice CT. As a result, the described embodiments of the present invention facilitate imaging a patient in a cost-effective and reliable manner.

Exemplary embodiments of imaging system methods and apparatus are described above in detail. The imaging system components illustrated are not limited to the specific embodiments described herein, but rather, components of each imaging system may be utilized independently and separately from other components described herein. For example, the imaging system components described above may also be used in combination with different imaging systems. A technical effect of the various embodiments of the systems and methods described herein include at least one of facilitating imaging a patient with images wherein instrument placement accuracy is enhanced.

While the invention has been described in terms of various specific embodiments, those skilled in the art will recognize that the invention can be practiced with modification within the spirit and scope of the claims.

What is claimed is:

1. An imaging system comprising a multi-slice detector, a processor coupled to said multi-slice detector, and a display configured to display reconstructed images, said processor configured to:
receive a plurality of multi-slice scan data;
identify at least a portion of an instrument in at least one slice of the multi-slice scan data; and
display the identified instrument portion with an indicator associated with the at least one slice.

2. An imaging system in accordance with claim 1 wherein said indicator is at least one of a color, a shading, and a pattern.

3. An imaging system in accordance with claim 1 wherein said instrument is a needle-like instrument.

4. An imaging system in accordance with claim 1 wherein said instrument is a biopsy needle.

5. An imaging system in accordance with claim 1 wherein said processor is further programmed to:
display an image of a region of interest using multiple slices of the multi-slice scan data combined into a relatively thicker slice image; and
display the instrument concurrently on the image using each slice of the plurality of multi-slice scan data.

6. An imaging system in accordance with claim 1 wherein said processor is further programmed to:
display an image of a region of interest using multiple slices of the multi-slice scan data combined into a relatively thicker slice image in a first viewing area;
display the instrument concurrently on the image using each slice of the plurality of multi-slice scan data, each portion of the instrument positioned in a respective slice being displayed using an indicator associated with that slice; and
display the region of interest using a single slice of the multi-slice scan data in a second viewing area concurrently with the display of the first viewing area; and
display the instrument in the second viewing area using each slice of the plurality of multi-slice scan data, each portion of the instrument positioned in a respective slice being displayed using an indicator associated with that slice.

7. An imaging system in accordance with claim 6 wherein said processor is further programmed to scroll through selected slices of the multi-slice scan data in the second viewing area.

8. An imaging system in accordance with claim 7 wherein said processor is further programmed to receive an input from a user indicative of a selected slice to display in the second viewing area.

9. An imaging system in accordance with claim 1 wherein said processor is further programmed to:
analyze each slice of the multi-slice scan data; and
identify at least a portion of the instrument included in each slice.

10. An imaging system in accordance with claim 1 wherein said processor is further programmed to automatically identify the portion of the instrument included in each slice using at least one of image threshold detection based on a CT value of the instrument, an image analysis, and a preprocessed sinogram data analysis based on predetermined instrument entrance and target locations.

11. An imaging system in accordance with claim 1 wherein said processor is further programmed to display the identified instrument portion with a predetermined color spectrum such that a color is associated with each of the at least one slice.

12. An imaging system in accordance with claim 1 wherein said processor is further programmed to display the identified instrument portion with a predetermined color spectrum such that a different color is associated with each adjacent slice.

13. A computer system configured to:
receive a plurality of multi-slice scan data; and
identify at least a portion of a needle-like instrument positioned in at least one slice of the multi-slice scan data with an indicator associated with the slice.

14. A computer system in accordance with claim 13 further configured to associate an indicator including at least one of a color, a shading, and a pattern with each slice of a multi-slice image of a region of interest.

15. A computer system in accordance with claim 13 further configured to apply the indicator associated with the slice to the identified instrument portion in the slice.

16. A computer system in accordance with claim 13 further configured to apply the indicator including at least one of a color, a shading, a texture, and a pattern.

17. A computer system in accordance with claim 13 further configured to display the identified instrument portion with an indicator associated with the at least one slice.

18. A computer system in accordance with claim 13 further configured to:
display an image of a region of interest using multiple slices of the multi-slice scan data combined into a relatively thicker slice image; and
display the instrument concurrently on the image using each slice of the multi-slice scan data.

19. A computer system in accordance with claim 13 further configured to:
display an image of a region of interest using multiple slices of the multi-slice scan data combined into a relatively thicker slice image in a first viewing area;
display the identified instrument portion concurrently on the image using each slice of the plurality of multi-slice scan data, each portion of the identified instrument portion positioned in a respective slice being displayed using an indicator associated with that slice; and
display the region of interest using a single slice of the multi-slice scan data in a second viewing area concurrently with the display of the first viewing area; and
display the identified instrument portion in the second viewing area using each slice of the multi-slice scan data, each portion of the instrument positioned in a respective slice being displayed using an indicator associated with that slice.

20. A computer system in accordance with claim 13 further configured to scroll through selected slices of the multi-slice scan data in the second viewing area.

21. A computer system in accordance with claim 13 further configured to receive an input from a user indicative of a selected slice to display in the second viewing area.

22. A computer system in accordance with claim 13 further configured to:
analyze each slice of the multi-slice scan data; and
identify at least a portion of the instrument included in each slice.

23. A computer system in accordance with claim 13 further configured to automatically identify the identified instrument portion included in each slice using at least one of image threshold detection based on a CT value of the instrument, an image analysis, and a preprocessed sinogram data analysis based on predetermined instrument entrance and target locations.

24. A method of displaying an instrument in a region of interest comprising:
associating an indicator including at least one of a color, a shading, and a pattern with each slice of a multi-slice image of a region of interest;
identifying at least a portion of an instrument in at least one slice; and
applying the indicator associated with the slice, to the identified instrument portion in that slice.

25. A method in accordance with claim 24 further comprising receiving a plurality of multi-slice scan data.

26. A method in accordance with claim 24 further comprising displaying the identified instrument portion with an indicator associated with the at least one slice.

27. A method in accordance with claim 24 wherein applying the indicator associated with the slice comprises applying at least one of a color, a shading, a texture, and a pattern.

28. A method in accordance with claim 24 further comprising:
displaying an image of a region of interest using multiple slices of the multi-slice scan data combined into a relatively thicker slice image; and
displaying the instrument concurrently on the image using each slice of the plurality of multi-slice scan data.

29. A method in accordance with claim 24 further comprising:
displaying an image of a region of interest using multiple slices of the multi-slice scan data combined into a relatively thicker slice image in a first viewing area;
displaying the identified instrument portion concurrently on the image using each slice of the plurality of multi-slice scan data, each portion of the instrument positioned in a respective slice being displayed using an indicator associated with that slice; and
displaying the region of interest using a single slice of the multi-slice scan data in a second viewing area concurrently with the display of the first viewing area; and
displaying the identified instrument portion in the second viewing area using each slice of the multi-slice scan data, each portion of the instrument positioned in a respective slice being displayed using an indicator associated with that slice.

30. A method in accordance with claim 29 further comprising scrolling through selected slices of the multi-slice scan data in the second viewing area.

31. A method in accordance with claim 30 further comprising receiving an input from a user indicative of a selected slice to display in the second viewing area.

32. A method in accordance with claim 24 wherein identifying at least a portion of an instrument in at least one slice comprises:
analyzing each slice of the multi-slice scan data; and
identifying a portion of the instrument included in each slice.

33. A method in accordance with claim 24 wherein identifying at least a portion of an instrument in at least one slice comprises automatically identifying the portion of the instrument included in each slice using at least one of image threshold detection based on a CT value of the instrument, an image analysis, and a preprocessed sinogram data analysis based on predetermined instrument entrance and target locations.

34. A method in accordance with claim 24 further comprising displaying the identified instrument portion with a predetermined color spectrum such that a color is associated with each of the at least one slice.

35. A method in accordance with claim 24 further comprising displaying the identified instrument portion with a predetermined color spectrum such that a different color is associated with each adjacent slice.

36. An imaging scanner comprising:
a data acquisition apparatus configured to acquire imaging data from a subject;
a monitor configured to display images reconstructed from the acquired imaging data; and
a computer programmed to:
acquire multiple slices of imaging data from the subject having an intracorporeal device positioned therein;
reconstruct a multi-slice image from the multiple slices of imaging data; and
cause the monitor to display the multi-slice image at a real-time frame rate while preserving information of a position of the intracorporeal device contained in the multiple slices of imaging data for observation by a human observer.

37. The imaging scanner of claim 36 wherein the computer is further programmed to:
acquire CT imaging data;
determine a position of a portion of the intracorporeal device positioned within a cavity of the subject from which the CT imaging data is acquired; and
cause the monitor to display the multi-slice image with pixels of the image corresponding to the portion of the intracorporeal device having at least one of a conspicuous color, shade, and pattern relative to other pixels in the multi-slice image.

38. The imaging scanner of claim 37 wherein the intracorporeal device has multiple sections and wherein the computer is further programmed to determine a respective position of each section of the intracorporeal device and assign at least one of a unique color, shade, and pattern to respective pixels of the multi-slice image.

39. The imaging scanner of claim 38 wherein the computer is further programmed to assign the at least one of a unique color, shade, and pattern to respective pixels of each section of the intracorporeal based on which slice of the multiple slices the section of the intracorporeal device was positioned in when the multiple slices of imaging data were acquired.

40. The imaging scanner of claim 37 wherein the computer is further programmed to cause the monitor to display a single composite image from the multiple slices of imaging data overlayed with one of a multi-color image, multi-shade image, and a multi-pattern image of the intracorporeal device that is updated at the real-frame rate as the intracorporeal device is repositioned within the cavity.

41. The imaging scanner of claim 37 wherein the computer is further programmed to:
determine a position of a tip of the intracorporeal device;
cause the monitor to display a single slice image for a slice location defined by the position of the tip of the intracorporeal device; and
assign at least one of a conspicuous color, shade, or pattern to those pixels of the image corresponding to CT imaging data acquired from the tip.

42. The imaging scanner of claim 41 wherein the single slice image is selected to lie in a plane of anatomy targeted for imaging.

43. The imaging scanner of claim 37 wherein the computer is further programmed to:
compare CT values of a slice of CT imaging data to a threshold; and
determine portions of the slice of CT imaging data corresponding to the intracorporeal device from the comparison.

44. The imaging scanner of claim 36 wherein the real-time frame rate includes 10 frames per second.

45. The imaging scanner of claim 36 wherein the intracorporeal device is a fluoroscopy needle or a biopsy needle.

46. A method of tracking an invasive instrument relative to a target using an imaging system that includes a movable patient table and a multi-slice detector array to automatically move the scan plane of the imaging system within the Z coverage area of the multi-slice detector array, the method comprising:
determining an intracorporeal trajectory of the instrument;
displaying a tip of the instrument in at least one of a plurality of adjacent slices; and
translating a patient table when the tip reaches a substantial extent of the Z coverage area.

47. A method in accordance with claim 46 wherein determining an intracorporeal trajectory of the instrument comprises:
locating a display cursor on each of the invasive instrument tip entry point and the target to determine a planned instrument trajectory; and
positioning a movable patient table such that the instrument tip appears on an image slice using the display cursor locations.

48. A method in accordance with claim 46 wherein determining an intracorporeal trajectory of the instrument comprises adjusting the initial entry angle of the instrument using a guide.

49. A method in accordance with claim 48 wherein the guide includes at least one of a laser, a calipers, and a light.

50. A method in accordance with claim 48 wherein adjusting the initial entry angle of the instrument using a guide comprises acquiring at least one of a continuous and a tap scan of the instrument during insertion into the patient.

51. A method in accordance with claim 48 wherein determining an intracorporeal trajectory of the instrument comprises determining the trajectory using at least two images wherein the images are based on data acquired by more than one detector row.

52. A method in accordance with claim 46 wherein displaying a tip of the instrument in at least one of a plurality of adjacent slices comprises verifying an XY angle of the instrument using information from an image including the insertion point.

53. A method in accordance with claim 46 wherein displaying a tip of the instrument in at least one of a plurality of adjacent slices comprises verifying an angle relative to the Z-axis using information from an image including the insertion point and an image including the tip.

54. A method in accordance with claim 46 wherein displaying a tip of the instrument in at least one of a plurality of adjacent slices comprises calculating a movement direction of the instrument using a current image and a previous image.

55. A method in accordance with claim 54 wherein calculating a movement direction of the instrument using a current image and a temporally-adjacent previous image comprises using a current image and a temporally-spaced previous image.

56. A method in accordance with claim 54 further comprising predicting a location of an appearance of the tip in an image using the initial entry angle, the calculated needle movement direction and a slice thickness.

57. A method in accordance with claim 54 further comprising predicting a location of an appearance of the tip in an adjacent image if the needle is completely included in only one image, using the tip position in the only one image.

58. A method in accordance with claim 56 wherein predicting a location of an appearance of the tip in an image comprises verifying the area corresponding to the predicted appearance point on an image using a current image and a previous image.

59. A method in accordance with claim 58 wherein verifying the area corresponding to the predicted appearance point on an image comprises observing a substantial density change within the predicted appearance area.

60. A method in accordance with claim 58 wherein verifying the area corresponding to the predicted appearance point on an image comprises observing a density change for several consecutive reconstructed images.

61. A method in accordance with claim 58 wherein verifying the area corresponding to the predicted appearance point on an image comprises for a substantially rigid, straight instrument with a relatively small angle with respect to the Z-axis, observing a density change for two consecutive reconstructed images.

62. A method in accordance with claim 58 wherein verifying the area corresponding to the predicted appearance point on an image comprises for a curved instrument, observing a density change using a relatively thinner slice thickness and a relatively larger predicted appearance area.

63. A method in accordance with claim 59 wherein observing a substantial density change within the predicted appearance area comprises generating images of the tip using slices that are shifter one slice in the direction of movement of the tip.

64. A method in accordance with claim 59 wherein observing a substantial density change within the predicted appearance area comprises translating an upper beam collimator of the imaging system in the Z-direction an amount corresponding to one slice in the direction of movement of the tip.

65. A method in accordance with claim 46 wherein displaying a tip of the instrument in at least one of a plurality of adjacent slices comprises determining in real-time, the instrument trajectory is substantially coincident with the predetermined trajectory.

66. A method in accordance with claim 65 wherein determining in real-time, the instrument trajectory is substantially coincident with the predetermined trajectory comprises transmitting an alarm if the instrument deviates from the predetermined trajectory by a selectable position threshold.

67. A method in accordance with claim 46 wherein translating a patient table when the tip reaches a substantial extent of the Z coverage area comprises when the tip reaches a selectable limit of the Z-axis coverage of the multi-slice detector array, warning the user that movement of the patient table is necessary to maintain the tip within the viewing capability of the imaging system.

68. A method in accordance with claim 46 wherein translating a patient table when the tip reaches a substantial extent of the Z coverage area comprises when the tip is predicted to exit the last slice of the multi-slice detector array, warning the user that movement of the patient table is necessary to maintain the tip within the viewing capability of the imaging system.

69. A method in accordance with claim 46 further comprising:
determining a gantry tilt angle that facilitates reducing a dose to the user during the scan; and
tilting the gantry to perform the scan.
